(19) United States
(12) Patent Application Publication
(10) Pub. No.: US 2011/0130386 A1
(43) Pub. Date: Jun. 2, 2011

(54) DNA-PK INHIBITORS

(76) Inventors: Mark Frigerio, Cambridge (GB); Heather Mary Ellen Duggan, Cambridge (GB); Marc Geoffrey Hummersone, Cambridge (GB); Keith Allan Menear, Cambridge (GB); Niall Morrison Barr Martin, Cambridge (GB); Celine Cano, Newcastle-upon-Tyne (GB); Roger John Griffin, Newcastle-upon-Tyne (GB); Bernard Thomas Golding, Newcastle-upon-Tyne (GB); Ian Robert Hardcastle, Newcastle-upon-Tyne (GB)

(21) Appl. No.: 12/989,510

(22) PCT Filed: Apr. 24, 2009
(86) PCT No.: PCT/GB09/01041

§ 371 Date: Feb. 9, 2011

Related U.S. Application Data
(60) Provisional application No. 61/047,917, filed on Apr. 25, 2008.

(51) Int. Cl. A61K 31/551; C07D 413/14; C07D 243/06; C12N 5/071; A61K 31/5355; A61K 31/55; A61P 35/00

(52) U.S. Cl. 514/217.05; 544/146; 544/141; 544/129; 544/121; 544/85; 540/598; 540/575; 435/325; 514/231.5; 514/232.2; 514/235.8; 514/218

(57) ABSTRACT

A compound of formula (I) wherein: R1 and R2 are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms; and R^{N1} and R^{N2} are selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms.

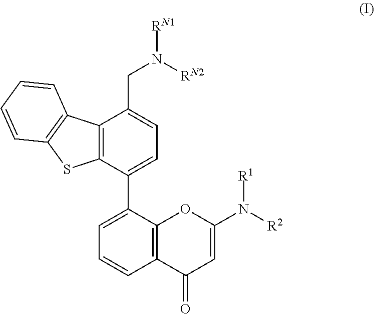

### The present invention relates to compounds which act as DNA-PK inhibitors, their use and synthesis.

The DNA-dependent protein kinase (DNA-PK) is a nuclear serine/threonine protein kinase that is activated upon association with DNA. Biochemical and genetic data have revealed this kinase to be composed of a large catalytic subunit, termed DNA-PKcs, and a regulatory component termed Ku. DNA-PK has been shown to be a crucial component of both the DNA double-strand break (DSB) repair machinery and the V(D)J recombination apparatus. In addition, recent work has implicated DNA-PK components in a variety of other processes, including the modulation of chromatin structure and telomere maintenance (Smith, G. C. M. and Jackson, S. P., Genes and Dev., 13, 916-934 (1999)).

DNA DSBs are regarded as the most lethal lesion a cell can encounter. To combat the serious threats posed by DNA DSBs, eukaryotic cells have evolved several mechanisms to mediate their repair. In higher eukaryotes, the predominant of these mechanisms is DNA non-homologous end-joining (NHEJ), also known as illegitimate recombination. DNA-PK plays a key role in this pathway. Increased DNA-PK activity has been demonstrated both in vitro and in vivo and correlates with the resistance of tumour cells to IR and bifunctional alkylating agents (Muller C., et al., Blood, 92, 2213-2219 (1998), Sirzen F., et al., Eur. J. Cancer, 35, 111-116 (1999)). Therefore, increased DNA-PK activity has been proposed as a cellular and tumour resistance mechanism. Hence, inhibition of DNA-PK with a small molecule inhibitor may prove efficacious in tumours where over-expression is regarded as a resistance mechanism.

It also has been previously found that the PI 3-kinase inhibitor LY294002:

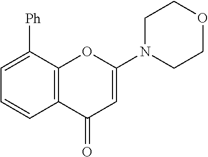

is able to inhibit DNA-PK function in vitro (Izzard, R. A., et al., Cancer Res., 59, 2581-2586 (1999)). The IC₅₀ (concentration at which 50% of enzyme activity is lost) for LY294002 towards DNA-PK is, at ˜1 μM, the same as that for PI 3-kinase. Furthermore it has been shown that LY294002 is also able to weakly sensitise cells to the effects of IR (Rosenzweig, K. E., et al., Clin. Cancer Res., 3, 1149-1156 (1999)).

WO 03/024949 describes a number of classes of compounds useful as DNA-PK inhibitors, including 2-amino-chromen-4-ones of the general structure:

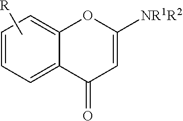

of which:

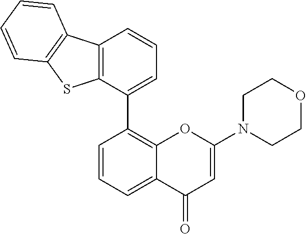

was one example. This compound exhibited an IC₅₀ of 10-12 nM and an SER of 1.3 (see below for methods).

WO 2006/032869 describes compounds useful as DNA-PK inhibitors, including 2-amino-chromen-4-ones of the general structure:

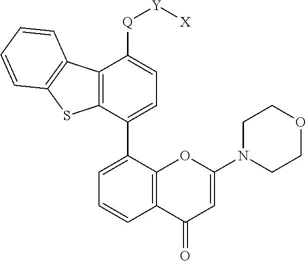

wherein:

Q represents —NH—C(═O)— or —O—, Y is an optionally substituted C₁₋₅ alkylene group and X is selected from H, or a thioether or amino group.

Given the involvement of DNA-PK in DNA repair processes, and that small molecule inhibitors have been shown to radio- and chemo-sensitise mammalian cells in culture, an application of specific DNA-PK inhibitory drugs would be to act as agents that will enhance the efficacy of both cancer chemotherapy and radiotherapy. DNA-PK inhibitors may also prove useful in the treatment of retroviral mediated diseases. For example it has been demonstrated that loss of DNA-PK activity severely represses the process of retroviral integration (Daniel R, et al., Science, 284, 644-7 (1999)).

The present inventors have now discovered further compounds which exhibit similar or improved levels of DNA-PK inhibition, whilst possessing other useful properties for use as active pharmaceuticals, in particular improved solubility and cellular efficacy. Some of the compounds of the present invention also show good solubility in both aqueous media and phosphate buffer solution—enhanced solubility may be of use in formulation the compounds for administration by an IV route, or for oral formulations (e.g. liquid and small tablet forms) for paediatric use. The oral bioavailablity of the compounds of the present invention may be enhanced.

Accordingly, the first aspect of the invention provides a compound of formula I:

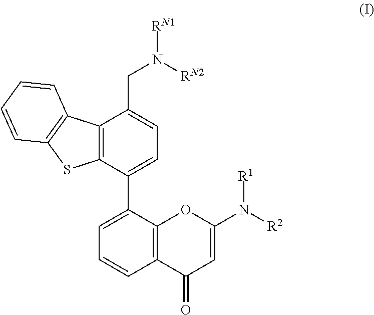

wherein:

R¹ and R² are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;

R^{N1} and R^{N2} are selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms.

A second aspect of the invention provides a composition comprising a compound of the first aspect and a pharmaceutically acceptable carrier or diluent.

A third aspect of the invention provides a compound of the first aspect for use in a method of therapy.

A fourth aspect of the invention provides for the use of a compound of the first aspect in the preparation of a medicament for treating a disease ameliorated by the inhibition of DNA-PK. The fourth aspect of the invention also provides a compound of the first aspect for use in the method of treatment of a disease ameliorated by the inhibition of DNA-PK.

It is preferred that in the fourth aspect the compounds of the first aspect selectivity inhibit the activity of DNA-PK compared to PI 3-kinase and/or ATM. Selectivity is an important issue as inhibition of other PI 3-kinase family members may lead to unwanted side-effects associated with the loss of function of those enzymes.

In particular in the fourth aspect of the invention, the compounds may be:
(a) used as, or in the preparation of a medicament for use as an adjunct in cancer therapy or for potentiating tumour cells for treatment with ionising radiation or chemotherapeutic agents; or
(b) used to treat, or in the preparation of a mediacament for the treatment of, retroviral mediated diseases.

A further aspect of the invention provides an active compound as described herein for use in a method of treatment of the human or animal body, preferably in the form of a pharmaceutical composition.

Another aspect of the invention provides a method of inhibiting DNA-PK in vitro or in vivo, comprising contacting a cell with an effective amount of an active compound as described herein.

Definitions

C₁₋₇ alkyl: The term “C₁₋₇ alkyl”, as used herein, pertains to a monovalent moiety obtained by removing a hydrogen atom from a C₁₋₇ hydrocarbon compound having from 1 to 7 carbon atoms, which may be aliphatic or alicyclic, or a combination thereof, and which may be saturated, partially unsaturated, or fully unsaturated.

Examples of saturated linear C₁₋₇ alkyl groups include, but are not limited to, methyl, ethyl, n-propyl, n-butyl, and n-pentyl (amyl).

Examples of saturated branched C₁₋₇ alkyl groups include, but are not limited to, iso-propyl, iso-butyl, sec-butyl, tert-butyl, and neo-pentyl.

Examples of saturated alicyclic C₁₋₇ alkyl groups (also referred to as “C₃₋₇cycloalkyl” groups) include, but are not limited to, groups such as cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of unsaturated C₁₋₇ alkyl groups which have one or more carbon-carbon double bonds (also referred to as “C₂₋₇alkenyl” groups) include, but are not limited to, ethenyl (vinyl, —CH═CH₂), 2-propenyl (allyl, —CH—CH═CH₂), isopropenyl (—C(CH₃)═CH₂), butenyl, pentenyl, and hexenyl.

Examples of unsaturated C₁₋₇ alkyl groups which have one or more carbon-carbon triple bonds (also referred to as “C₂₋₇alkynyl” groups) include, but are not limited to, ethynyl (ethinyl) and 2-propynyl (propargyl).

Examples of unsaturated alicyclic (carbocyclic) C₁₋₇ alkyl groups which have one or more carbon-carbon double bonds (also referred to as “C₃₋₇cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

C₃₋₂₀ heterocyclyl: The term “C₃₋₂₀ heterocyclyl”, as used herein, pertains to a monovalent moiety obtained by removing a hydrogen atom from a ring atom of a C₃₋₂₀ heterocyclic compound, said compound having one ring, or two or more rings (e.g., spiro, fused, bridged), and having from 3 to 20 ring atoms, atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms. Ring heteroatoms may preferably be selected from the group consisting of O, N, S and P. “C₃₋₂₀” denotes ring atoms, whether carbon atoms or heteroatoms.

Examples of C₃₋₂₀ heterocyclyl groups having one nitrogen ring atom include, but are not limited to, those derived from aziridine, azetidine, pyrrolidines (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine.

Examples of C₃₋₂₀ heterocyclyl groups having one oxygen ring atom include, but are not limited to, those derived from oxirane, oxetane, oxolane (tetrahydrofuran), oxole (dihydrofuran), oxane (tetrahydropyran), dihydropyran, pyran (C₆), and oxepin. Examples of substituted C₃₋₂₀ heterocyclyl groups include sugars, in cyclic form, for example, furanoses and pyranoses, including, for example, ribose, lyxose, xylose, galactose, sucrose, fructose, and arabinose.

Examples of C₃₋₂₀ heterocyclyl groups having one sulphur ring atom include, but are not limited to, those derived from thiirane, thietane, thiolane (tetrahydrothiophene), thiane (tetrahydrothiopyran), and thiepane.

Examples of C₃₋₂₀ heterocyclyl groups having two oxygen ring atoms include, but are not limited to, those derived from dioxolane, dioxane, and dioxepane.

Examples of C₃₋₂₀ heterocyclyl groups having two nitrogen ring atoms include, but are not limited to, those derived from imidazolidine, pyrazolidine (diazolidine), imidazoline, pyrazoline (dihydropyrazole), and piperazine.

Examples of C₃₋₂₀ heterocyclyl groups having one nitrogen ring atom and one oxygen ring atom include, but are not limited to, those derived from tetrahydrooxazole, dihydrooxazole, tetrahydroisoxazole, dihydroisoxazole, morpholine, tetrahydrooxazine, dihydrooxazine, and oxazine.

Examples of C₃₋₂₀ heterocyclyl groups having one oxygen ring atom and one sulphur ring atom include, but are not limited to, those derived from oxathiolane and oxathiane (thioxane).

Examples of C₃₋₂₀ heterocyclyl groups having one nitrogen ring atom and one sulphur ring atom include, but are not limited to, those derived from thiazoline, thiazolidine, and thiomorpholine.

Other examples of C₃₋₂₀heterocyclylgroups include, but are not limited to, oxadiazine and oxathiazine.

Examples of heterocyclyl groups which additionally bear one or more oxo (═O) groups, include, but are not limited to, those derived from:

C₅ heterocyclics, such as furanone, pyrone, pyrrolidone (pyrrolidinone), pyrazolone (pyrazolinone), imidazolidone, thiazolone, and isothiazolone;

C₆ heterocyclics, such as piperidinone (piperidone), piperidinedione, piperazinone, piperazinedione, pyridazinone, and pyrimidinone (e.g., cytosine, thymine, uracil), and barbituric acid;

fused heterocyclics, such as oxindole, purinone (e.g., guanine), benzoxazolinone, benzopyrone (e.g., coumarin);

cyclic anhydrides (—C(═O)—O—C(═O)— in a ring), including but not limited to maleic anhydride, succinic anhydride, and glutaric anhydride;

cyclic carbonates (—O—C(═O)—O— in a ring), such as ethylene carbonate and 1,2-propylene carbonate;

imides (—C(═O)—NR—C(═O)— in a ring), including but not limited to, succinimide, maleimide, phthalimide, and glutarimide;

lactones (cyclic esters, —O—C(═O)— in a ring), including, but not limited to, β-propiolactone, γ-butyrolactone, δ-valerolactone (2-piperidone), and ε-caprolactone;

lactams (cyclic amides, —NR—C(═O)— in a ring), including, but not limited to, β-propiolactam, γ-butyrolactam (2-pyrrolidone), δ-valerolactam, and ε-caprolactam;

cyclic carbamates (—O—C(═O)—NR— in a ring), such as 2-oxazolidone;

cyclic ureas (—NR—C(═O)—NR— in a ring), such as 2-imidazolidone and pyrimidine-2,4-dione (e.g., thymine, uracil).

C₅₋₂₀ aryl: The term “C₅₋₂₀ aryl”, as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups”, in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), naphthacene (C₁₈), and pyrene (C₁₆).

Examples of aryl groups which comprise fused rings, one of which is not an aromatic ring, include, but are not limited to, groups derived from indene and fluorene.

Alternatively, the ring atoms may include one or more heteroatoms, including but not limited to oxygen, nitrogen, and sulphur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

Examples of C₅₋₂₀ heterocyclic groups (some of which are C₅₋₂₀ heteroaryl groups) which comprise fused rings, include, but are not limited to, C₉ heterocyclic groups derived from benzofuran, isobenzofuran, indole, isoindole, purine (e.g., adenine, guanine), benzothiophene, benzimidazole; C₁₀ heterocyclic groups derived from quinoline, isoquinoline, benzodiazine, pyridopyridine, quinoxaline; C₁₃ heterocyclic groups derived from carbazole, dibenzothiophene, dibenzofuran; C₁₄ heterocyclic groups derived from acridine, xanthene, phenoxathiin, phenazine, phenoxazine, phenothiazine.

The above C₁₋₇ alkyl, C₃₋₂₀ heterocyclyl and C₅₋₂₀ arylgroups whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

C₁₋₇ alkoxy: —OR, wherein R is a C₁₋₇ alkyl group. Examples of C₁₋₇ alkoxy groups include, but are not limited to, —OCH₃ (methoxy), —OCH₂CH₃ (ethoxy) and —OC(CH₃)₃ (tert-butoxy).

Oxo (keto, -one): ═O. Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam (2-pyrrolidone), δ-valerolactam, and ε-caprolactam.

Imino (imine): ═NR, wherein R is an imino substituent, for example, hydrogen, C₁₋₇ alkyl group, a C₃₋₂₀heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COON.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇alkyl group. Examples of acyloxy groups include, but are not limited to, —OC(═O)CH₃(acetoxy), —OC(═O)CH₂CH₃, —OC(═O)C(CH₃)₃, —OC(═O)Ph, and —OC(═O)CH₂Ph.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl and phthalimidyl:

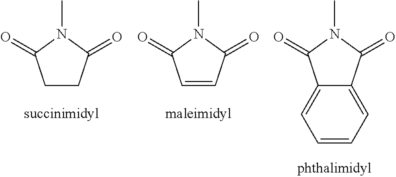

Acylureido: —N(R¹)C(O)NR²C(O)R³ wherein R¹ and R² are independently ureido substituents, for example, hydrogen, a C₁₋₇ alkyl group, a C₃₋₂₀ heterocyclyl group, or a C₅₋₂₀ aryl group, preferably hydrogen or a C₁₋₇ alkyl group. R³ is an acyl group as defined for acyl groups. Examples of acylureido groups include, but are not limited to, —NHCONHC(O)H, —NHCONMeC(O)H, —NHCONEtC(O)H, —NHCONMeC(O)Me, —NHCONEtC(O)Et, —NMeCONHC(O)Et, —NMeCONHC(O)Me, —NMeCONHC(O)Et, —NMeCONMeC(O)Me, —NMeCONEtC(O)Et, and —NMeCONHC(O)Ph.

Carbamate: —NR¹—C(O)—OR² wherein R¹ is an amino substituent as defined for amino groups and R² is an ester group as defined for ester groups. Examples of carbamate groups include, but are not limited to, —NH—C(O)—O-Me, —NMe-C(O)—O-Me, —NH—C(O)—O-Et, —NMe-C(O)—O-t-butyl, and —NH—C(O)—O-Ph.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Tetrazolyl: a five membered aromatic ring having four nitrogen atoms and one carbon atom,

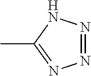

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group.

Amidine: —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. An example of an amidine group is —C(═NH)NH₂.

Carbazoyl (hydrazinocarbonyl): —C(O)—NN—R¹ wherein R¹ is an amino substituent as defined for amino groups. Examples of azino groups include, but are not limited to, —C(O)—NN—H, —C(O)—NN-Me, —C(O)—NN-Et, —C(O)—NN-Ph, and —C(O)—NN—CH₂-Ph.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃, —S(═O)₂C₄F₉ (nonaflyl), —S(═O)₂CH₂CF₃ (tresyl), —S(═O)₂Ph (phenylsulfonyl), 4-methylphenylsulfonyl (tosyl), 4-bromophenylsulfonyl (brosyl), and 4-nitrophenyl (nosyl).

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ and —OS(═O)₂CH₂CH₃.

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Sulfamyl: —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh,

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅. A special class of sulfonamino groups are those derived from sultams—in these groups one of R¹ and R is a C₅₋₂₀ aryl group, preferably phenyl, whilst the other of R¹ and R is a bidentate group which links to the C₅₋₂₀ aryl group, such as a bidentate group derived from a C₁₋₇ alkyl group. Examples of such groups include, but are not limited to:

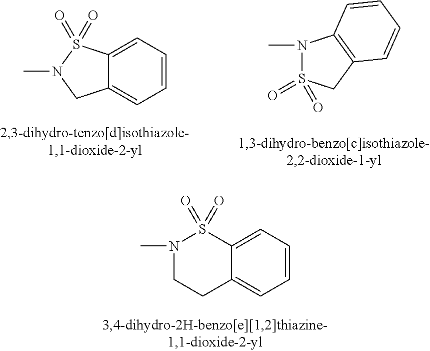

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

In many cases, substituents may themselves be substituted. For example, a C₁₋₇ alkoxy group may be substituted with, for example, a C₁₋₇ alkyl (also referred to as a C₁₋₇ alkyl-C₁₋₇alkoxy group), for example, cyclohexylmethoxy, a C₃₋₂₀ heterocyclyl group (also referred to as a C₅₋₂₀ aryl-C₁₋₇alkoxy group), for example phthalimidoethoxy, or a C₅₋₂₀ aryl group (also referred to as a C₅₋₂₀aryl-C₁₋₇alkoxy group), for example, benzyloxy.

Includes Other Forms

Included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

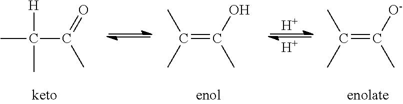

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule.

See, for example, Protective Groups in Organic Synthesis (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO$).

For example, a carboxylic acid group may be protected as an ester for example, as: an C₁₋₇ alkyl ester (e.g. a methyl ester; a t-butyl ester); a C₁₋₇ haloalkyl ester (e.g., a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₇ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Selective Inhibition

‘Selective inhibition’ means the inhibition of one enzyme to a greater extent than the inhibition of one or more other enzymes. This selectivity is measurable by comparing the concentration of a compound required to inhibit 50% of the activity (IC₅₀) of one enzyme against the concentration of the same compound required to inhibit 50% of the activity (IC₅₀) of the other enzyme (see below). The result is expressed as a ratio. If the ratio is greater than 1, then the compound tested exhibits some selectivity in its inhibitory action.

The compounds of the present invention preferably exhibit a selectivity of greater than 3, 10, 20 or 50 against DNA-PK over PI 3-kinase.

The compounds of the present invention preferably exhibit a selectivity of greater than 5, 10, 50 or 100 against DNA-PK over ATM.

It is preferred that the IC₅₀ values used to assess selectivity are determined using the methods described in WO 03/024949, which is herein incorporated by reference.

### Further Embodiments

R^{N1} and R^{N2}

In some embodiments, R^{N1} is H.

In these embodiments, R^{N2} may be selected from hydrogen, an optionally substituted C₁₋₇ alkyl group, C₃₋₂₀ heterocyclyl group, or C₅₋₂₀ aryl group. In some embodiments, R^{N2} may be selected from an optionally substituted C₁₋₇ or C₁₋₄ alkyl group (e.g. methyl, ethyl, propylene, cylcopropyl, pentyl) and C₅₋₂₀ or C₅₋₆ aryl group (e.g. phenyl, isoxazolyl, triazolyl, pyrrolyl). R^{N2} may also be a C₃₋₂₀ or C₃₋₇ heterocyclyl group (e.g. tetrahydrothiophenyl, pyrrolidinyl, piperidinyl, dihydrothiazolyl).

In these embodiments, the C₁₋₇ or C₁₋₄ alkyl group may be unsubstituted, or optionally substituted by a group selected from a C₃₋₇ heterocyclic group (e.g tetrahydrofuranyl, N-methylpyrolidinyl, N-ethylpyrolidinyl, N-methylpiperidinyl), a C₅₋₇ aryl group (e.g. phenyl, pyridyl, imadzolyl, thiazolyl, pyrazolyl, furanyl, methylopyrazolyl, dimethylisoxazolyl, pyrrolyl, cyanopyrrolyl), hydroxy, ether (e.g. C₁₋₇alkyl ether, itself optionally substituted, e.g. with hydroxy), amino and diC₁₋₄ alkylamino (e.g. dimethylamino). The optional substituent may also include cyano and carboxy,

In these embodiments, the C₅₋₂₀ or C₅₋₆ aryl group may be unsubstituted, or optionally substituted by a group selected from C₁₋₄ alkyl (e.g. methyl) and cyano. The C₅₋₂₀ or C₅₋₆ aryl group may be selected from phenyl, methyl-isoxazolyl, methyl-triazolyl, cyanopyrrolyl and triazolyl.

In these embodiments, the C₃₋₂₀ or C₃₋₇ heterocyclyl group may be unsubstituted or optionally substituted by one or more groups seledted from: oxo (in particular on a sulphr ring atom to form a dioxosulfur ring atom) and C₁₋₄ alkyl (e.g. methyl). The C₃₋₂₀ or C₃₋₇ heterocyclyl group may be selected from tetrahydrothiophenyl-dioxide, methylpyrrolidyl, dihydrothiazolyl and methylpiperidinyl.

In some embodiments, R^{N1} is C₁₋₇ or C₁₋₄ alkyl (e.g. methyl, ethyl).

In these embodiments, R^{N2} may be selected from hydrogen, an optionally substituted C₁₋₇ alkyl group, C₃₋₂₀ heterocyclyl group, or C₅₋₂₀ aryl group. In some embodiments, R^{N2} may be an optionally substituted C₁₋₇ or C₁₋₄ alkyl group (e.g. methyl, ethyl).

In these embodiments, the C₁₋₇ or C₁₋₄ alkyl group may be unsubstituted, or optionally substituted by a group selected from a C₃₋₇ heterocyclic group (e.g tetrahydrofuranyl, N-methylpyrolidinyl, N-ethylpyrolidinyl, N-methylpiperidinyl), a C₅₋₇ aryl group (e.g. phenyl, pyridyl, imadzolyl, thiazolyl, pyrazolyl, furanyl, methylopyrazolyl, dimethylisoxazolyl, pyrrolyl, cyanopyrrolyl), hydroxy, ether (e.g. C₁₋₇alkyl ether, itself optionally substituted, e.g. with hydroxy), amino and diC₁₋₄ alkylamino (e.g. dimethylamino). In particular, the C₁₋₇ or C₁₋₄ alkyl group may be optionally substituted by a group selected from a C₅₋₇ aryl group (e.g. phenyl) and diC₁₋₄ alkylamino (e.g. dimethylamino). The optional substituent may also include cyano and carboxy,

In these embodiments, the C₅₋₂₀ or C₅₋₆ aryl group may be unsubstituted, or optionally substituted by a group selected from C₁₋₄ alkyl (e.g. methyl),and cyano. The C₅₋₂₀ or C₅₋₆ aryl group may be selected from phenyl, methyl-isoxazolyl, methyl-triazolyl, cyanopyrrolyl and triazolyl.

In these embodiments, the C₃₋₂₀ or C₃₋₇ heterocyclyl group may be unsubstituted or optionally substituted by one or more groups seledted from: oxo (in particular on a sulphr ring atom to form a dioxosulfur ring atom) and C₁₋₄ alkyl (e.g. methyl). The C₃₋₂₀ or C₃₋₇ heterocyclyl group may be selected from tetrahydrothiophenyl-dioxide, methylpyrrolidyl, dihydrothiazolyl and methylpiperidinyl.

In some embodiments, R^{N1} and R^{N2} form, along with the nitrogen atom to which they are attached, a heterocyclic ring having from 4 to 8 atoms, this may form part of a C₄₋₂₀ heterocyclyl group defined above (except with a minimum of 4 ring atoms), which must contain at least one nitrogen ring atom. In some embodiments, R^{N1} and R^{N2} form, along with the nitrogen atom to which they are attached, a heterocyclic ring having 5, 6 or 7 atoms.

Single rings having one nitrogen atom include azetidine, azetidine, pyrrolidine (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine; two nitrogen atoms include imidazolidine, pyrazolidine (diazolidine), imidazoline, pyrazoline (dihydropyrazole), and piperazine; one nitrogen and one oxygen include tetrahydrooxazole, dihydrooxazole, tetrahydroisoxazole, dihydroisoxazole, morpholine, tetrahydrooxazine, dihydrooxazine, and oxazine; one nitrogen and one sulphur include thiazoline, thiazolidine, and thiomorpholine.

Rings of particular interest in the present invention are those containing no or one heteroatom in addition to the nitrogen, where the additional heteroatom is usually selected from nitrogen, oxygen and sulphur. Thus, a group of embodiments of interest are those where R^{N1} and R^{N2} together with the nitrogen atom to which they are attached form an optionally substituted heterocylic ring selected from: pyrrolidine, piperidine, piperazine, morpholine, homopiperidine and homopiperazine. Other groups of interest include thiomorpholine, isoxaolidine and [1,2]oxazinane.

As mentioned above, these heterocyclic groups may themselves be substituted. The optional substituents may be selected from the group comprising: hydroxy; C₁₋₇ or C₁₋₄ alkoxy (e.g. methoxy); C₁₋₇ or C₁₋₄ alkyl (e.g. methyl, ethyl, propyl), which may itself be further substituted by a group selected from hydroxy and methoxy; C₅₋₇ aryl (e.g. phenyl), which may itself be further substituted by a group selected from hydroxy and methoxy; acyl, where the acyl substituent may be C₁₋₄ alkyl (e.g. methyl, cyclopropyl); and diC₁₋₄ alkylamino (e.g. dimethylamino). The substituents may also include fluoro, oxo. The C₁₋₇ or C₁₋₄ alkyl substituent groups may themselves be substituted by fluorine (e.g the group may be CF₃).

These substituents may be on any available ring atom. if the ring comprises a further nitrogen atom, the substitution may be on this nitrogen ring atom.

If the ring formed by R^{N1} and R^{N2} and the nitrogen atom to which they are bound is pyrrolidine, then the optional substituents may be selected from the group comprising: hydroxy; and diC₁₋₄ alkylamino (e.g. dimethylamino). The optional substituents may also be selected from C₁₋₇ or C₁₋₄ alkyl (e.g. methyl, ethyl, propyl), which may itself be further substituted by hydroxy; and halo.

If the ring formed by R^{N1} and R^{N2} and the nitrogen atom to which they are bound is piperidine, then the optional substituents may be selected from the group comprising: hydroxy; C₁₋₇ or C₁₋₄ alkoxy (e.g. methoxy); C₁₋₇ or C₁₋₄ alkyl (e.g. methyl, ethyl, propyl) may itself be further substituted by hydroxy; and diC₁₋₄ alkylamino (e.g. dimethylamino). The C₁₋₇ or C₁₋₄ alkyl substituent groups may themselves be substituted by fluorine (e.g the group may be CF₃). The optional substituents may also be halo (e.g. fluoro).

If the ring formed by R^{N1} and R^{N2} and the nitrogen atom to which they are bound is piperazine, then the optional substituents may be selected from the group comprising: C₁₋₇ or C₁₋₄ alkyl (e.g. methyl, ethyl, propyl), which may itself be further substituted by a group selected from hydroxy and methoxy; C₅₋₇ aryl (e.g. phenyl), which may itself be further substituted by methoxy; and acyl, where the acyl substituent may be C₁₋₄ alkyl (e.g. methyl, cyclopropyl). The optional substituents may also be oxo.

If the ring formed by R^{N1} and R^{N2} and the nitrogen atom to which they are bound is morpholine, then the optional substituents may be C₁₋₇ or C₁₋₄ alkyl (e.g. methyl, ethyl, propyl), which may itself be further substituted by hydroxy.

If the ring formed by R^{N1} and R^{N2} and the nitrogen atom to which they are bound is homopiperidine, then the ring may be unsubstituted.

If the ring formed by R^{N1} and R^{N2} and the nitrogen atom to which they are bound is homopiperazine, then the optional substituents may be C₁₋₇ or C₁₋₄ alkyl (e.g. methyl, ethyl, propyl), which may itself be further substituted by methoxy.

In some embodiments, R^{N1}, R^{N2} and the nitrogen atom to which they are attached form a group represented by formula (II):

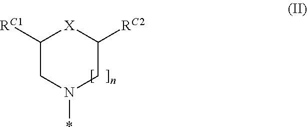

wherein X is O, S, CHR^{X} or NR^{X};

n is 0, 1 or 2;

R^{C1} and R^{C2} are independently selected from H and methyl; and

R^{X} is selected from the group consisting of H, and optionally substituted C₁₋₇ alkyl, C₃₋₂₀ heterocyclyl, C₅₋₂₀ aryl, acyl, ester and sulfonyl.

n

In some embodiments, n is 1. In other embodiments, n is 2.

X

In some embodiments, X is N. In other embodiments, X is CH.

R^{C1} and R^{C2}

If R^{C1} and R^{C2} are both methyl, R^{X} may be selected from C₁₋₄ alkyl and H. In some of these embodiments, R^{X} may be H.

In some embodiments, R^{C1} and R^{C2} are both H.

R^{X}

In some embodiments when X is N, R^{X} is selected from the group consisting of H, and optionally substituted C₁₋₇ alkyl, C₅₋₂₀ aryl, acyl, ester and sulfonyl. In some of these embodiments, R^{X} is selected from the group consisting of H and optionally substituted C₁₋₇ alkyl and sulfonyl.

In some embodiments, the C₁₋₇ alkyl group may be a C₁₋₄ alkyl group, and may be, for example, selected from methyl, ethyl and propyl. The optional susbtitutents for the C₁₋₇ alkyl group may include, but are not limited to, C₅₋₂₀ aryl (e.g. phenyl), C₃₋₂₀ heterocyclyl (e.g. morpholino, tetrahydrofuranyl), halo (e.g. fluoro, chloro), hydroxy, ether (e.g. C₁₋₇ alkoxy), acyl (e.g. C₁₋₇ alkylcarbonyl), carboxy, ester (e.g. C₁₋₇ alkyl ester), acyloxy, amido, acylamido, amino, cyano and C₃₋₇ cycloalkyl (e.g. cyclopropyl). In some of these embodiments, the optional substituents on the C₁₋₇ alkyl group may be selected from ether (e.g. C₁₋₇ alkoxy), acyl (e.g. C₁₋₇ alkylcarbonyl), cyano and C₃₋₇ cycloalkyl (e.g. cyclopropyl).

In some embodiments, the C₅₋₂₀ aryl group may be a C₅₋₇ aryl group, and may be, for example, selected from phenyl and pyridyl. The optional susbtitutents for the C₅₋₂₀ aryl group may include, but are not limited to, C₁₋₇ alkyl (e.g. methyl, ethyl), C₃₋₂₀ heterocyclyl (e.g. morpholino), halo (e.g. fluoro, chloro), hydroxy, ether (e.g. C₁₋₇ alkoxy), acyl (e.g. C₁₋₇ alkylcarbonyl), carboxy, ester (e.g. C₁₋₇ alkyl ester), acyloxy, amido, acylamido, amino, cyano and C₃₋₇ cycloalkyl (e.g. cyclopropyl).

In some embodiments, the acyl group may have as the acyl substituent a C₁₋₇ alkyl group (e.g. methyl) or a C₃₋₂₀ heterocyclyl group (e.g. tetrhydrofuranyl).

In some embodiments, the ester group may have as the ester substituent a C₁₋₇ alkyl or C₁₋₄ alkyl group (e.g. t-butyl).

In some embodiments, the sulfonyl group may have as the sulfone substituent a C₁₋₇ alkyl or C₁₋₄ alkyl group (e.g. methyl, ethyl).

In some embodiments when X is CH, R^{X} is C₃₋₂₀ heterocyclyl. R^{X} may also be acyl.

In some of these embodiments, the C₃₋₂₀ heterocylyl group is a C₅₋₇ heterocyclyl group (e.g. morpholino).

In some of these embodiments, the acyl group may have as the acyl substituent a C₁₋₇ alkyl or C₁₋₄ alkyl group (e.g. methyl, ethyl).

The substituent groups may themselves by substituted as described above. For example, if one of the groups described is substituted by an ether group (e.g. C₁₋₇ alkoxy), and then that group may itself be susbsituted by a hydroxy, C₁₋₇ alkyl or ether (e.g. C₁₋₇ alkoxy) group.

R¹ and R²

In compounds of formula I, when R¹ and R² form, along with the nitrogen atom to which they are attached, a heterocyclic ring having from 4 to 8 atoms, this may form part of a C₄₋₂₀ heterocyclyl group defined above (except with a minimum of 4 ring atoms), which must contain at least one nitrogen ring atom. It is preferred that R¹ and R² form, along with the nitrogen atom to which they are attached, a heterocyclic ring having 5, 6 or 7 atoms, more preferably 6 ring atoms.

Single rings having one nitrogen atom include azetidine, azetidine, pyrrolidine (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine; two nitrogen atoms include imidazolidine, pyrazolidine (diazolidine), imidazoline, pyrazoline (dihydropyrazole), and piperazine; one nitrogen and one oxygen include tetrahydrooxazole, dihydrooxazole, tetrahydroisoxazole, dihydroisoxazole, morpholine, tetrahydrooxazine, dihydrooxazine, and oxazine; one nitrogen and one sulphur include thiazoline, thiazolidine, and thiomorpholine.

Preferred rings are those containing one heteroatom in addition to the nitrogen, and in particular, the preferred heteroatoms are oxygen and sulphur. Thus preferred groups include morpholino, thiomorpholino, thiazolinyl. Preferred groups without a further heteroatom include pyrrolidino.

The most preferred groups are morpholino and thiomorpholino.

As mentioned above, these heterocyclic groups may themselves be substituted; a preferred class of substituent is a C₁₋₇ alkyl group. When the heterocyclic group is morpholino, the substituent group or groups are preferably methyl or ethyl, and more preferably methyl. A sole methyl substituent is most preferably in the 2 position.

As well as the single ring groups listed above, rings with bridges or cross-links are also envisaged. Examples of these types of ring where the group contains a nitrogen and an oxygen atom are:

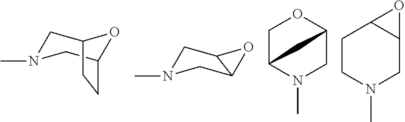

These are named 8-oxa-3-aza-bicyclo[3.2.1]oct-3-yl, 6-oxa-3-aza-bicyclo[3.1.0]hex-3-yl, 2-oxa-5-aza-bicyclo[2.2.1]hept-5-yl, and 7-oxa-3-aza-bicyclo[4.1.0]hept-3-yl, respectively.

General Synthesis Methods

Compounds of formula I:

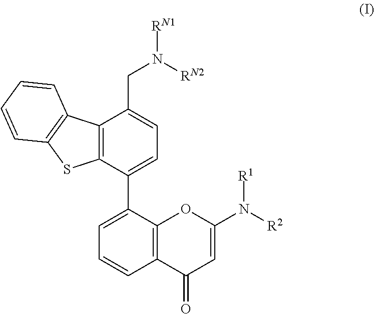

can be synthesised by coupling compounds of formulae 1 and 2:

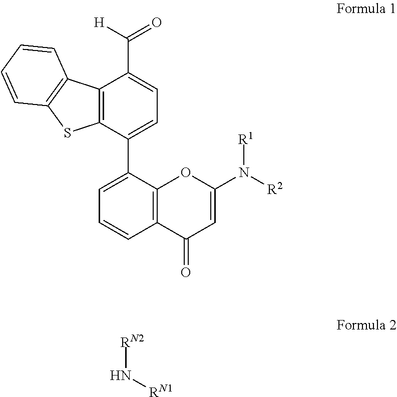

vy Lewis acid mediated reductive amination. The Lewis Acid may be, for example, titanium (VI) isopropoxide, and the reducing agent may be, for example, sodium borohydride.

Compounds of formula 2 can be derived from compounds of formula 3:

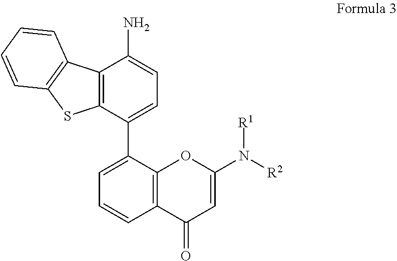

by diazotisation (using, for example, t-butyl nitrite), followed by palladocarbonylation, using a palladium catalyst, for example, palladium (II) acetate.

### Use of Compounds of the Invention

The present invention provides active compounds, specifically, active substituted dibeznothiophenyl, amino-chromen-4-ones.

The term “active”, as used herein, pertains to compounds which are capable of inhibiting DNA-PK activity, and specifically includes both compounds with intrinsic activity (drugs) as well as prodrugs of such compounds, which prodrugs may themselves exhibit little or no intrinsic activity.

One assay which may be used in order to assess the DNA-PK inhibition offered by a particular compound is described in the examples below.

The present invention further provides a method of inhibiting DNA-PK inhibition in a cell, comprising contacting said cell with an effective amount of an active compound, preferably in the form of a pharmaceutically acceptable composition. Such a method may be practised in vitro or in vivo.

For example, a sample of cells (e.g. from a tumour) may be grown in vitro and an active compound brought into contact with said cells in conjunction with agents that have a known curative effect, and the enhancement of the curative effect of the compound on those cells observed.

The present invention further provides active compounds which inhibit DNA-PK activity as well as methods of methods of inhibiting DNA-PK activity comprising contacting a cell with an effective amount of an active compound, whether in vitro or in vivo.

Active compounds may also be used as cell culture additives to inhibit DNA-PK, for example, in order to sensitize cells to known chemotherapeutic agents or ionising radiation treatments in vitro.

Active compounds may also be used as part of an in vitro assay, for example, in order to determine whether a candidate host is likely to benefit from treatment with the compound in question.

The invention further provides active compounds for use in a method of treatment of the human or animal body. Such a method may comprise administering to such a subject a therapeutically-effective amount of an active compound, preferably in the form of a pharmaceutical composition.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “therapeutically-effective amount” as used herein, pertains to that amount of an active compound, or a material, composition or dosage from comprising an active compound, which is effective for producing some desired therapeutic effect, commensurate with a reasonable benefit/risk ratio.

The term “adjunct” as used herein relates to the use of active compounds in conjunction with known therapeutic means. Such means include cytotoxic regimes of drugs and/or ionising radiation as used in the treatment of different cancer types. Examples of adjunct anti-cancer agents that could be combined with compounds from the invention include, but are not limited to, the following: alkylating agents: nitrogen mustards, mechlorethamine, cyclophosphamide, ifosfamide, melphalan, chlorambucil: Nitrosoureas: carmustine (BCNU), lomustine (CCNU), semustine (methyl-CCNU), ethylenimine/methylmelamine, thriethylenemelamine (TEM), triethylene thiophosphoramide (thiotepa), hexamethylmelamine (HMM, altretamine): Alkyl sufonates; busulfan; Triazines, dacarbazine (DTIC): Antimetabolites; folic acid analogs, methotrexate, trimetrexate, pyrimidine analogs, 5-fluorouracil, fluorodeoxyuridine, gemcitabine, cytosine arabinoside (AraC, cytarabine), 5-azacytidine, 2,2′-difluorodeoxycytidine: Purine analogs; 6-mercaptopurine, 6-thioguanine, azathioprine, 2′-deoxycoformycin (pentostatin, erythrohydroxynonyladenine (EHNA), fludarabine phosphate, 2-Chlorodeoxyadenosine (cladribine, 2-CdA): Topoisomerase I inhibitors; camptothecin, topotecan, irinotecan, rubitecan: Natural products; antimitotic drugs, paclitaxel, vinca alkaloids, vinblastine (VLB), vincristine, vinorelbine, Taxotere™ (docetaxel), estramustine, estramustine phosphate; epipodophylotoxins, etoposide, teniposide: Antibiotics; actimomycin D, daunomycin (rubidomycin), doxorubicin (adriamycin), mitoxantrone, idarubicin, bleomycins, plicamycin (mithramycin), mitomycin C, dactinomycin: Enzymes; L-asparaginase, RNAse A: Biological response modifiers; interferon-alpha, IL-2, G-CSF, GM-CSF: Differentiation Agents; retinoic acid derivatives: Radiosensitizers;, metronidazole, misonidazole, desmethylmisonidazole, pimonidazole, etanidazole, nimorazole, RSU 1069, EO9, RB 6145, SR4233, nicotinamide, 5-bromodeozyuridine, 5-iododeoxyuridine, bromodeoxycytidine: Platinium coordination complexes; cisplatin, carboplatin: Anthracenedione; mitoxantrone, AQ4N Substituted urea, hydroxyurea; Methylhydrazine derivatives, N-methylhydrazine (MIH), procarbazine; Adrenocortical suppressant, mitotane (o.p′-DDD), aminoglutethimide: Cytokines; interferon (α, β, γ), interleukin; Hormones and antagonists; adrenocorticosteroids/antagonists, prednisone and equivalents, dexamethasone, aminoglutethimide; Progestins, hydroxyprogesterone caproate, medroxyprogesterone acetate, megestrol acetate; Estrogens, diethylstilbestrol, ethynyl estradiol/equivalents; Antiestrogen, tamoxifen; Androgens, testosterone propionate, fluoxymesterone/equivalents; Antiandrogens, flutamide, gonadotropin-releasing hormone analogs, leuprolide; Nonsteroidal antiandrogens, flutamide; EGFR inhibitors, VEGF inhibitors; Proteasome inhibitors.

Cancer

The present invention provides active compounds which are anticancer agents or adjuncts for treating cancer. One of ordinary skill in the art is readily able to determine whether or not a candidate compound treats a cancerous condition for any particular cell type, either alone or in combination.

Examples of cancers include, but are not limited to, lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma and leukemias.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [HerceptinT], the anti-EGFR antibody panitumumab, the anti erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (AvastinT) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

Formulations

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavoured basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethyihexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 pg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

The following are examples are provided solely to illustrate the present invention and are not intended to limit the scope of the invention, as described herein.

Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

General Experimental Details

Chemicals were purchased from the Aldrich Chemical Company, Lancaster Synthesis Ltd and Acros Organics (Fisher Scientific UK Ltd). THF was freshly distilled from sodium/benzophenone. Methanol and ethanol were distilled from magnesium/iodine. DCM was dried by distillation over phosphorus pentoxide. Acetone was dried by distillation over calcium hydride. All solvents not used immediately were stored over molecular sieves (4 Å, 3-5 mm beads), under nitrogen. Anhydrous DMF was obtained from Aldrich in SureSeal™ bottles. Triethylamine was dried by distillation over calcium hydride and stored over potassium hydroxide, under nitrogen.

Thin layer chromatography (TLC), was performed using Merck silica gel 60F₂₅₄ pre-coated on aluminium sheets which were subsequently dried and visualised using either short wave (254 nm) ultraviolet light or by treatment with either ninhydrin or sulphuric acid then vanillin. ‘Flash’ column chromatography was carried out at medium pressure using Davisil silica gel (40-63 μm).

Melting points were determined using a Stuart Scientific SMP3 apparatus and are uncorrected. ¹H and ¹³C nuclear magnetic resonance (NMR) spectra were obtained using a Bruker Spectrospin AC 300E spectrometer (¹H 300 MHz or ¹³C 75 MHz) or a Bruker Spectrospin AC 500E spectrometer (¹H 500 MHz or ¹³C 125 MHz). Chemical shifts are reported in parts per million (δ) downfield of tetramethylsilane using residual solvent peaks as internal standards. Multiplicities are indicated by s (singlet), d (doublet), t (triplet), q (quartet), m (multiplet), br (broad) or combinations thereof. LC/MS spectra were obtained using a Micromass Platform instrument running in positive or negative ion electrospray mode. Separation was achieved using a C18 column (50×4.6 mm; Supelco Discovery or Waters Symmetry) and a 15 minute gradient elution of 0.05% formic acid and methanol (10-90%). IR spectra were recorded on a Bio-Rad FTS 3000MX diamond ATR as a neat sample.

Compounds were purified either using a mass-directed LC-MS system or a UV directed System.

Mass-Directed LC-MS System

This uses a Waters ZQ mass spectrometer, Waters 600 pump and Waters 2700 sample manager. Mobile phase A—0.1% formic acid in water, Mobile phase B—0.1% formic acid in acetonitrile, Flow rate 20 ml/min., Gradient: 5% B to 75% B over 15 minutes, then to 100% B over 1 minute, hold for 1 minute. Column: Phenomenex Gemini C18, 5 um, 110A, Axia, 20 50 mm×21.2 mm

UV-Directed System

This uses Gilson 305 and 306 pumps, with a Gilson 155 uv/vis detector, Gilson 215 injector/collector. Mobile phase A—0.1% formic acid in water, mobile phase B—0.1% formic acid in acetonitrile, Flow rate 6 ml/min, Gradient: 10% B for 3 minutes, then to 95% B over 16 minutes, hold for 5 minutes. Column: Hichrom ACE 5 um C18. 250 mm×10 mm.

### Example 1

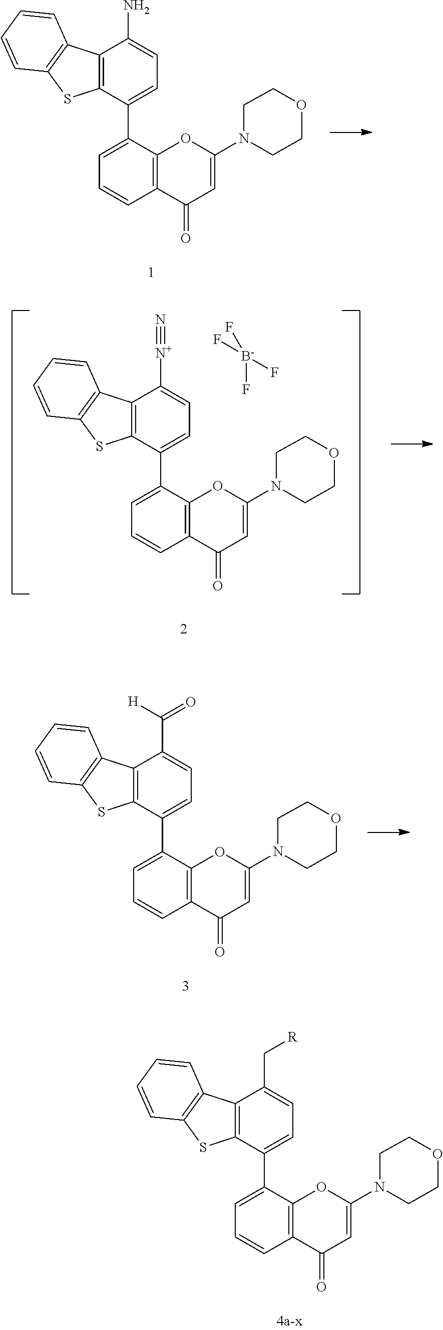

### (a) 4-(2-Morpholin-4-yl-4-oxo-4H-chromen-8-yl)-dibenzothiophene-1-diazonium tetrafluoroborate (2)

To a 0.1 M solution of 8-(1-amino-dibenzothiophen-4-yl)-2-morpholin-4-yl-chromen-4-one (1) (1 equiv) in EtOH was added HBF₄ (54% in Et₂O, 300 equiv) in a slow stream. After stirring the brown mixture, at room temperature, for 15 minutes the reaction was cooled to 0° C. t-Butyl nitrite (3.6 equiv) was then added in a dropwise fashion to the reaction, while ensuring the solution temperature did not exceed 5° C. After stirring at 0° C. for 16 hours, Et₂O (1 reaction volume) was added. The resultant precipitate was removed by filtration, and washed with cold Et₂O (1 reaction volume), dried under vacuum at room to give the desired product in a suitably pure form to be used without any further manipulation. Yield: 99% yield; Purity: 96%; m/z (LC-MS, ESP): 440.4 (weak)[M+H]⁺ R/T=3.45 mins.

### (b) 4-(2-Morpholin-4-yl-4-oxo-4H-chromen-8-yl)-dibenzothiophene-1-carbaldehyde (3)

To a suspension of 4-(2-morpholin-4-yl-4-oxo-4H-chromen-8-yl)-dibenzothiophene-1-diazonium tetrafluoroborate (2) (1 equiv) in MeCN/Et2O (0.5 M) was added triisopropyl silane (1.1 equiv) and palladium (II) acetate (0.02 equiv). The reaction vessel was sealed and subjected to pressurised carbon dioxide (10 bar) while stirring at room temperature for 6 hours. After this time the reaction was diluted with CH₂Cl₂ (1 reaction volume) and washed with saturated NaHCO₃ (0.5 reaction volume) and brine (0.5 reaction volume). The organic extract was dried (MgSO₄), filtered and concentrated in vacuo to give the desired product as a pale orange powder. Yield: 56%; Purity: 99%; m/z (LC-MS, ESP): 422.5[M+H]⁺ R/T=4.35 mins.

### (c) Library Synthesis

To a (0.01 M) solution of 4-(2-morpholin-4-yl-4-oxo-4H-chromen-8-yl)-dibenzothiophene-1-carbaldehyde (1 equiv) in toluene was added titanium(VI)isopropoxide (1 equiv) and the appropriate amine (3 equiv). The reaction vessel was sealed and subjected to microwave radiation (110° C., low absorption setting) for 15 minutes whereupon the reaction was allowed to cool to room temperature and sodium borohydride (3 equiv) added. After striiring the mixture for 16 hours, it was quenched by dropwise addition of water. The bi-phasic mixture was extracted using CH₂Cl₂ (3×1 reaction volume). The combined organic fractions were then dried (MgSO₄), filtered and concentrated in vacuo before being purified by preparative HPLC.

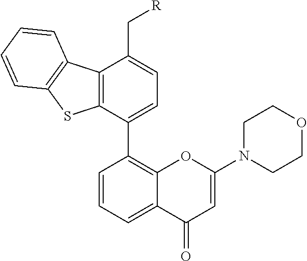

| | R | Purity | RT | [M + H]⁺ |
|---|---|---|---|---|
| 4a | 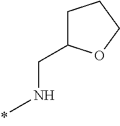 | 99 | 4.16 | 527.3 |
| 4b | 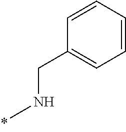 | 98 | 4.36 | 533.4 |
| 4c | 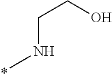 | 99 | 3.89 | 487.3 |
| 4d | 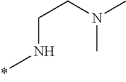 | 98 | 3.71 | 514.3 |
| 4e | 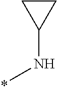 | 99 | 3.93 | 531.3 |
| 4f | 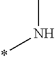 | 96 | 4.08 | 483.3 |
| 4g | 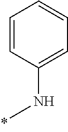 | 99 | 3.94 | 457.3 |
| 4h | 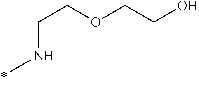 | 97 | 6.54 | 519.3 |
| 4i | 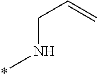 | 91 | 4.10 | 483.3 |
| 4j | 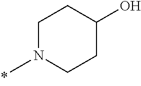 | 95 | 3.94 | 527.4 |
| 4k | 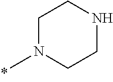 | 99 | 4.16 | 512.4 |
| 4l | 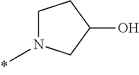 | 99 | 3.93 | 513.4 |
| 4m | 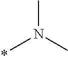 | 97 | 3.97 | 471.3 |
| 4n | 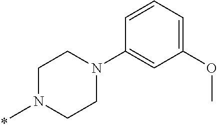 | 98 | 5.09 | 618.5 |
| 4o | 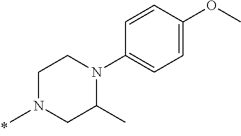 | 99 | 4.97 | 632.5 |
| 4p | 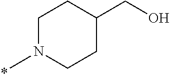 | 98 | 3.98 | 541.4 |
| 4q | 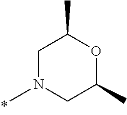 | 80 | 4.63 | 541.4 |
| 4r | 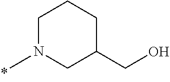 | 98 | 4 | 541.4 |
| 4s | 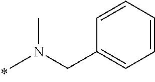 | 99 | 4.52 | 547.4 |
| 4t | 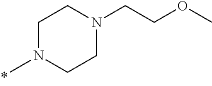 | 99 | 4.3 | 570.4 |
| 4u | 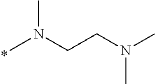 | 96 | 4.16 | 528.4 |
| 4v | 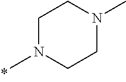 | 98 | 4.22 | 526.4 |
| 4x | 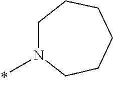 | 78 | 4.27 | 525.4 |
| 4y | 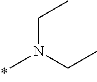 | 98 | 4.11 | 499.4 |
| 4z | 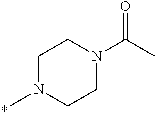 | 98 | 4.29 | 554.4 |
| 4aa | 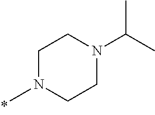 | 97 | 4.3 | 554.4 |
| 4ab | 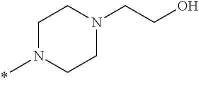 | 99 | 4.14 | 556.4 |
| 4ac | 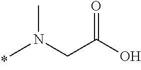 | 80 | 4.04 | 515.3 |
| 4ad | 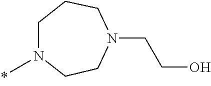 | 99 | 4.08 | 570.4 |
| 4ae | 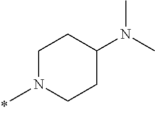 | 97 | 3.74 | 554.4 |
| 4af | 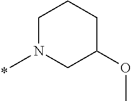 | 99 | 4.17 | 541.4 |
| 4ag | 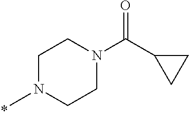 | 84 | 4.51 | 580.4 |
| 4ah | 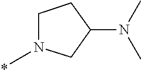 | 96 | 4.12 | 540.4 |
| 4ai | 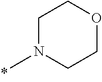 | 99 | 1.42 | 513.3 |
| 4aj | 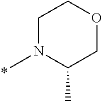 | 99 | 1.47 | 527.3 |
| 4ak | 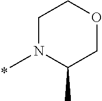 | 99 | 1.47 | 527.3 |
| 4al | 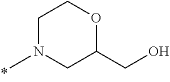 | 94 | 1.33 | 543.3 |
| 4am | 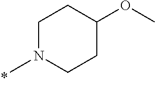 | 99 | 1.32 | 541.3 |
| 4an | 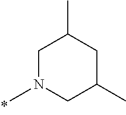 | 97 | 1.47 | 539.4 |
| 4ao | 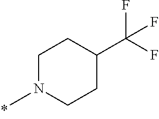 | 99 | 1.63 | 579.3 |
| 4ap | 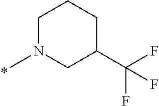 | 99 | 2.09 | 579.3 |
| 4ar | 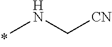 | 86 | 5.26 | 482.1 |
| 4as | 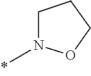 | 98 | 5.53 | 499.2 |
| 4at | 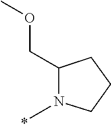 | 99 | 4.14 | 541.2 |
| 4au | 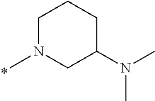 | 100 | 4.09 | 554.3 |
| 4av | 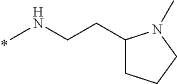 | 99 | 3.58 | 554.3 |
| 4aw | 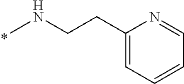 | 100 | 4.1 | 548.2 |
| 4ax | 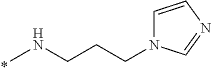 | 100 | 3.57 | 551.3 |
| 4ay | 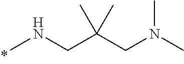 | 100 | 3.86 | 556.3 |
| 4az | 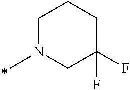 | 99 | 6.55 | 547.2 |
| 4ba | 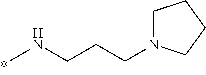 | 97 | 3.57 | 554.3 |
| 4bb | 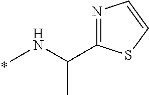 | 99 | 4.31 | 554.3 |
| 4bc | 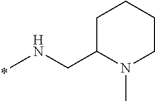 | 100 | 3.89 | 554.1 |
| 4bd | 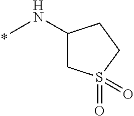 | 98 | 4.24 | 561 |
| 4be | 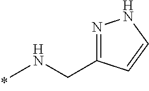 | 100 | 3.91 | 523.1 |
| 4bf | 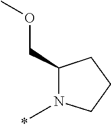 | 96 | 4.14 | 541.1 |
| 4bg | 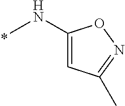 | 90 | 5.47 | 524 |
| 4bh | 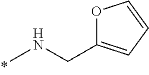 | 99 | 4.13 | 523.1 |
| 4bi | 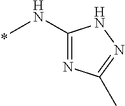 | 97 | 4.12 | 524 |
| 4bj | 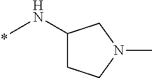 | 100 | 4.03 | 540.1 |
| 4bk | 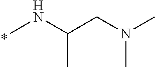 | 100 | 9.95 | 528.2 |
| 4bl | 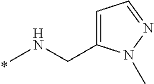 | 100 | 3.99 | 537.2 |
| 4bm | 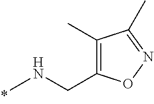 | 89 | 5.57 | 538.1 |
| 4bn | 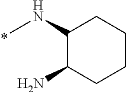 | 92 | 4.1 | 540.1 |
| 4bm | 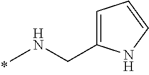 | 97 | 4.18 | 522.1 |
| 4bo | 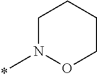 | 99 | 6.62 | 513.3 |
| 4bp | 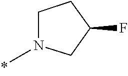 | 99 | 4.07 | 515 |
| 4bq | 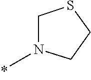 | 97 | 6.29 | 515 |
| 4br | 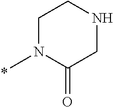 | 99 | 4.72 | 526.1 |
| 4bs | 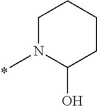 | 98 | 3.96 | 527.1 |
| 4bt | 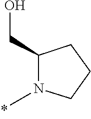 | 98 | 3.95 | 527.1 |
| 4bu | 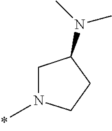 | 100 | 4.01 | 540.1 |
| 4bv | 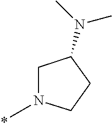 | 100 | 4 | 540.1 |
| 4bw | 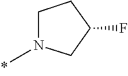 | 99 | 4.07 | 515 |
| 4bx | 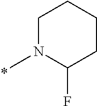 | 98 | 4.3 | 529.1 |
| 4by | 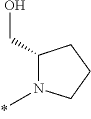 | 99 | 3.95 | 527.1 |
| 4bz | 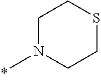 | 98 | 4.47 | 529.1 |
| 4ca | 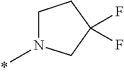 | 99 | 6.33 | 533 |
| 4cb | 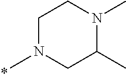 | 100 | 4.16 | 540.2 |
| 4cc | 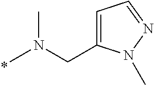 | 99 | 4.55 | 551.2 |
| 4cd | 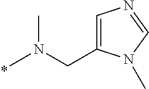 | 98 | 4.22 | 551.3 |
| 4ce | 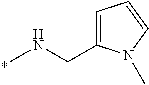 | 82 | 4.26 | 536.1 |
| 4cf | 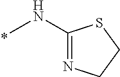 | 99 | 4.07 | 528.1 |
| 4cg | 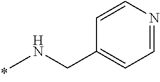 | 87 | 3.87 | 534.2 |
| 4ch | 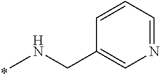 | 88 | 3.91 | 534.1 |
| 4ci | 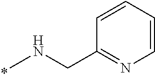 | 91 | 4.09 | 534.1 |
| 4cj | 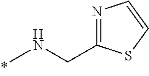 | 88 | 4.15 | 540 |
| 4ck | 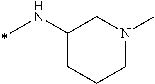 | 95 | 3.7 | 540.2 |
| 4cl | 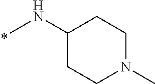 | 100 | 3.56 | 540.2 |
| 4cm | 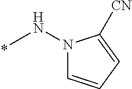 | 95 | 5.76 | 533.1 |
| 4cn | 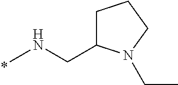 | 99 | 3.88 | 554.3 |
| 4co | 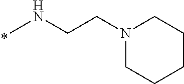 | 100 | 3.72 | 554.3 |
| 4cp | 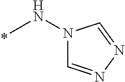 | 100 | 1.55 | 510.2 |
| 4cq | 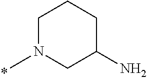 | 84 | 1.31 | 526.3 |
| 4cr | 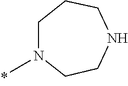 | 89 | 1.34 | 526.3 |

| | R | ¹H-NMR Spectrum: (CDCl₃, 300 MHz), δ (ppm) |
|---|---|---|
| 4aq | 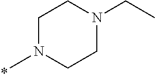 | 1.11 (3H, t, J = 7.2 Hz, CH₃); 2.59 (2H, q, J = 7.2 Hz, CH₂); 2.83-2.90 (4H, m, N—CH₂-piperazine); 3.01-3.06 (6H, m, CH₂ + N—CH₂-piperazine); 3.22-3.26 (4H, m, N—CH₂-morpholine); 3.42-3.45 (4H, m, O—CH₂-morpholine); 3.65-3.68 (4H, m, O—CH₂-morpholine); 4.06 (2H, s, CH₂); 5.52 (1H, s, CH-3); 7.26-7.80 (7H, m, Hₐᵣₒₘ); 8.19 (1H, dd, J = 1.5 and 7.8 Hz, Hₐᵣₒₘ); 8.24-8.27 (1H, m, Hₐᵣₒₘ). |
| 4cs | 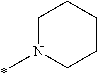 | 1.18-1.66 (6H, m, CH₂-piperidine); 2.99-3.01 (4H, m, N—CH₂-piperidine); 3.36-3.38 (4H, m, N—CH₂-morpholine); 3.40-3.44 (4H, m, O—CH₂-morpholine); 3.42 (2H, s, CH₂); 5.43 (1H, s, CH-3); 7.38-7.62 (4H, m, Hₐᵣₒₘ); 7.67 (1H, dd, J = 1.5 and 7.2 Hz, Hₐᵣₒₘ); 7.75-7.78 (1H, m, Hₐᵣₒₘ); 8.20 (1H, dd, J = 1.5 and 7.8 Hz, Hₐᵣₒₘ); 8.35-8.38 (1H, m, Hₐᵣₒₘ) |
| 4ct | 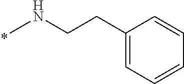 | 2.84-2.86 (2H, m, CH₂); 2.97-3.00 (4H, br s, N—CH₂-morpholine); 3.05-3.09 (2H, m, CH₂); 3.38-3.42 (4H, m, O—CH₂-morpholine); 4.40 (2H, s, CH₂); 5.43 (1H, s, CH-3); 7.14-7.46 (11H, m, Hₐᵣₒₘ); 7.66 (1H, dd, J = 1.8 and 7.8 Hz, Hₐᵣₒₘ); 7.73-7.76 (1H, m, Hₐᵣₒₘ); 8.19- 8.22 (1H, m, Hₐᵣₒₘ). |

### Biological Examples

DNA-PK Inhibition

In order to assess the inhibitory action of the compounds against DNA-PK in vitro, the following assay was used to determine IC₅₀ values.

Mammalian DNA-PK (500 ng/ml) was isolated from HeLa cell nuclear extract (Gell, D. and Jackson S. P., Nucleic Acids Res. 27:3494-3502 (1999)) following chromatography utilising Q-sepharose, S-sepharose and Heparin agarose. DNA-PK (250 ng) activity was measured at 30° C., in a final volume of 40 μl, in buffer containing 25 mM Hepes, pH7.4, 12.5 mM MgCl₂, 50 mM KCl, 1 mM DTT, 10% Glycerol, 0.1% NP-40 and 1 mg of the substrate GST-p53N66 (the amino terminal 66 amino acid resiudes of human wild type p53 fused to glutathione S-transferase) in polypropylene 96 well plates. To the assay mix, varying concentrations of inhibitor (in DMSO at a final concentration of 1%) were added. After 10 minutes of incubation, ATP was added to give a final concentration of 50 pM along with a 30 mer double stranded DNA oligonucleotide (final concentraion of 0.5 ng/ml) to initiate the reaction. After 1 hour with shaking, 150 μl of phosphate buffered saline (PBS) was added to the reaction and 5 μl then transferred to a 96 well opaque white plate containing 45 μl of PBS per well where the GSTp53N66 substrate was allowed to bind to the wells for 1 hour. To detect the phosphorylation event on the serine 15 residue of p53 elicited by DNA-PK a p53 phosphoserine-15 antibody (Cell Signaling Technology) was used in a basic ELISA procedure. An anti-rabbit HRP conjugated secondary antibody (Pierce) was then employed in the ELISA before the addition of chemiluminescence reagent. (NEN Renaissance) to detect the signal as measured by chemiluminescent counting via a TopCount NXT (Packard).

The enzyme activity for each compound is then calculated using the following equation:

$$\% Inhibition = 100 - ( \frac{( cpm of unknown - mean negative cpm ) \times 100}{( mean positive cpm - mean negative cpm )} )$$

The results are discussed below as IC₅₀ values (the concentration at which 50% of the enzyme activity is inhibited). These are determined over a range of different concentrations, normally from 10 μM down to 0.001 μM. Such IC₅₀ values are used as comparative values to identify increased compound potencies.

Survival Enhancement Ratio

The Survival Enhancement Ratio (SER) is a ratio of the enhancement of cell kill elicited by the DNA-PK inhibitor after 2 Grays of irradiation compared to unirradiated control cells. DNA-PK inhibitors were used at a concentration of 25, 50, 100 and/or 500 nM. Radiation was delivered by a Faxitron 43855D machine at a dose rate of 1 Gy pre minute The SER at 2 Gray irradiation was calculated from the formula:

$$SER = \frac{Cell survival in presence of DNA \text{-} PK inhibitor}{Cell survival of control cells} \times \frac{Cell survival after IR}{Cell survival after IR in presence of DNA \text{-} PK inhibitor}$$

The degree of cell killing was monitored by a standard clonogenic survival assay. Briefly, tissue culture treated 6-well plates were seeded with HeLa cells at an appropriate concentration to give 100-200 colonies per well and returned to the incubator in order to allow the cells to attach. Four hours later, compound or vehicle control was added to the cells. The cells were then incubated for 1 hour in the presence of inhibitor prior to irradiation at 2 Gray using a Faxitron 43855D cabinet X-ray machine. The cells were then incubated for a further 16 hours before the media was replaced with fresh media in the absence of DNA-PK inhibitor. After 8 days, colonies formed were fixed and stained with Giemsa (Sigma, Poole, UK) and scored using an automated colony counter (Oxford Optronics Ltd, Oxford, UK). The data was calculated as described above.

Results

The mean IC₅₀ of the compounds are given below:

| | IC₅₀ (μM) |
|---|---|
| 4a | 0.0312 |
| 4b | 0.248 |
| 4c | 0.0226 |
| 4d | 0.0126 |
| 4e | 0.0589 |
| 4f | 0.0168 |
| 4g | 0.0422 |
| 4h | 0.0248 |
| 4i | 0.883 |
| 4j | 0.508 |
| 4k | 0.00714 |
| 4l | 0.0303 |
| 4m | 0.293 |
| 4n | 0.975 |
| 4o | 0.502 |
| 4p | 0.0960 |
| 4q | 0.0833 |
| 4r | 0.124 |
| 4s | 0.942 |
| 4t | 0.00792 |
| 4u | 0.0273 |
| 4v | 0.00131 |
| 4x | 1.91 |
| 4y | 1.96 |
| 4z | 0.0779 |
| 4aa | 0.00175 |
| 4ab | 0.00139 |
| 4ac | 0.238 |
| 4ad | 0.00386 |
| 4ae | 0.177 |
| 4af | 0.232 |
| 4ag | 0.251 |
| 4ah | 0.00121 |
| 4ai | 0.102 |
| 4aj | 0.406 |
| 4ak | 0.912 |
| 4al | 0.0725 |
| 4am | 0.415 |
| 4an | 1.52 |
| 4ao | 5.43 |
| 4ap | 2.14 |
| 4aq | 0.00559 |
| 4ar | 0.0297 |
| 4as | 0.0689 |
| 4at | 0.884 |
| 4au | 0.0198 |
| 4av | 0.0486 |
| 4aw | 0.0696 |
| 4ax | 0.0265 |
| 4ay | 0.122 |
| 4az | 2.02 |
| 4ba | 0.0173 |
| 4bb | 0.777 |
| 4bc | 0.0105 |
| 4bd | 0.125 |
| 4be | 0.0917 |
| 4bf | 0.717 |
| 4bg | 0.299 |
| 4bh | 0.222 |
| 4bi | 0.0929 |
| 4bj | 0.00887 |
| 4bk | 0.0320 |
| 4bl | 0.0462 |
| 4bm | 1.16 |
| 4bn | 0.115 |
| 4bm | 0.00972 |
| 4bo | 2.07 |
| 4bp | 0.170 |
| 4bq | 0.433 |
| 4br | 0.0485 |
| 4bs | 0.127 |
| 4bt | 0.174 |
| 4bu | 0.00132 |
| 4bv | 0.00205 |
| 4bw | 0.438 |
| 4bx | 0.307 |
| 4by | 0.0986 |
| 4bz | 0.257 |
| 4ca | 1.01 |
| 4cb | 0.00374 |
| 4cc | 0.321 |
| 4cd | 0.0684 |
| 4ce | 0.291 |
| 4cf | 0.174 |
| 4cg | 0.139 |
| 4ch | 0.0985 |
| 4ci | 0.177 |
| 4cj | 0.193 |
| 4ck | 0.0161 |
| 4cl | 0.0472 |
| 4cm | 0.956 |
| 4cn | 0.0573 |
| 4co | 0.0625 |
| 4cp | 0.0946 |
| 4cq | 0.0361 |
| 4cr | 0.00667 |
| 4cs | 0.886 |
| 4ct | 0.530 |

The following compound exhbitied at SER of 1.5 or greater at 100 nM: 4a, 4c, 4d, 4f, 4g, 4h, 4k, 4l, 4t, 4u, 4v, 4aa, 4ab, 4ad, 4ah, 4aq, 4ar, 4au, 4bc, 4bj, 4bk, 4bu, 4bv, 4cb, 4ck, 4cl, 4cn, 4co, 4cr.

The following compound exhbitied at SER of 1.5 or greater at 50 nM: 4d, 4t, 4u, 4v, 4aa, 4ab, 4ad, 4ah, 4aq, 4bu.

1. A compound of formula I: 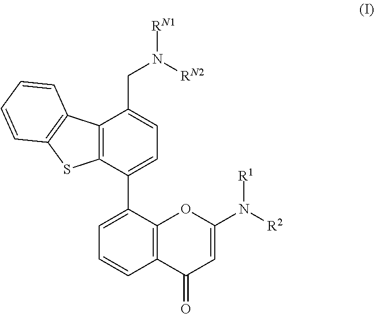
wherein:
R¹ and R² are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
R^{N1} and R^{N2} are selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms.

2. A compound according to claim 1, wherein R^{N1} is H and R^{N2} is selected from hydrogen, an optionally substituted C₁₋₇ alkyl group, C₃₋₂₀ heterocyclyl group, or C₅₋₂₀ aryl group.

3. A compound according to claim 2, wherein R^{N2} is selected from an optionally substituted C₁₋₄ alkyl group, C₃₋₇ heterocyclyl group and C₅₋₆ aryl group.

4. A compound according to claim 2, wherein the C₁₋₇ or C₁₋₄ alkyl group may be unsubstituted, or optionally substituted by a group selected from a C₃₋₇ heterocyclic group, a C₅₋₇ aryl group, hydroxy, carboxy, ether, cyano, amino and diC₁₋₄ alkylamino.

5. A compound according to claim 1, wherein R^{N1} is C₁₋₇ or C₁₋₄ alkyl, and R^{N2} is selected from hydrogen, an optionally substituted C₁₋₇ alkyl group, C₃₋₂₀ heterocyclyl group, or C₅₋₂₀ aryl group.

6. A compound according to claim 5, wherein R^{N2} is an optionally substituted C₁₋₇ or C₁₋₄ alkyl group.

7. A compound according to claim 5, wherein the C₁₋₇ or C₁₋₄ alkyl group is optionally substituted by a group selected from a C₃₋₇ heterocyclic group, a C₅₋₇ aryl group, hydroxy, carboxy, ether, cyano, amino and diC₁₋₄ alkylamino.

8. A compound according to claim 7, wherein the C₁₋₇ or C₁₋₄ alkyl group is optionally substituted by a group selected from a C₅₋₇ aryl group and diC₁₋₄ alkylamino.

9. A compound according to claim 1, wherein R^{N1} and R^{N2} form, along with the nitrogen atom to which they are attached, a heterocyclic ring having 5, 6 or 7 ring atoms.

10. A compound according to claim 9, wherein the heterocyclic ring is selected from: pyrrolidine, piperidine, piperazine, isoxaolodine, [1,2]oxazinane, morpholine, thiomorpholine, homopiperidine and homopiperazine.

11. A compound according to claim 1, wherein the optional substituents of the heterocyclic group are selected from the group comprising: hydroxy; fluoro; oxo; C₁₋₇ or C₁₋₄ alkoxy; C₁₋₇ or C₁₋₄ alkyl, which may further substituted by a group selected from hydroxy, fluoro and methoxy; C₅₋₇ aryl, which may itself be further substituted by a group selected from hydroxy and methoxy; acyl, where the acyl substituent is C₁₋₄ alkyl; and diC₁₋₄ alkylamino.

12. A compound according to claim 10, wherein the ring formed by R^{N1} and R^{N2} and the nitrogen atom to which they are bound is pyrrolidine, which is optionally substituted by one or more groups selected from: hydroxy; C₁₋₄ alkyl, and diC₁₋₄ alkylamino.

13. A compound according to claim 10, wherein the ring formed by R^{N1} and R^{N2} and the nitrogen atom to which they are bound is piperidine, which is optionally substituted by one or more groups selected from: hydroxy; halo, C₁₋₇ or C₁₋₄ alkoxy; C₁₋₇ or C₁₋₄ alkyl, which may itself be further substituted by hydroxy or fluoro; and diC₁₋₄ alkylamino.

14. A compound according to claim 10, wherein the ring formed by R^{N1} and R^{N2} and the nitrogen atom to which they are bound is piperazine, which is optionally substituted by one or more groups selected from: C₁₋₇ or C₁₋₄ alkyl, which may itself be further substituted by a group selected from hydroxy and methoxy; C₅₋₇ aryl, which may itself be further substituted by methoxy; oxo; and acyl, where the acyl substituent may be C₁₋₄ alkyl.

15. A compound according to claim 10, wherein the ring formed by R^{N1} and R^{N2} and the nitrogen atom to which they are bound is morpholine, which is optionally substituted by one or more C₁₋₇ or C₁₋₄ alkyl groups, which may itself be further substituted by hydroxy.

16. A compound according to claim 10, wherein the ring formed by R^{N1} and R^{N2} and the nitrogen atom to which they are bound is homopiperidine, which is unsubstituted.

17. A compound according to claim 10, wherein the ring formed by R^{N1} and R^{N2} and the nitrogen atom to which they are bound is homopiperazine, which is optionally substituted by one or more C₁₋₇ or C₁₋₄ alkyl groups, which may itself be further substituted by methoxy.

18. A compound according to claim 1, wherein R¹ and R² form, along with the nitrogen atom to which they are attached, a heterocyclic ring having 6 ring atoms.

19. A compound according to claim 18, wherein R¹ and R² form, along with the nitrogen atom to which they are attached, a group selected from morpholino, thiomorpholino, and thiazolinyl.

20. A compound according to claim 19, wherein R¹ and R² form, along with the nitrogen atom to which they are attached, morpholino.

21. A composition comprising a compound according to claim 1 and a pharmaceutically acceptable carrier or diluent.

22. (canceled)

23. (canceled)

24. A method of treatment of a disease ameliorated by the inhibition of DNA-PK, comprising administering an effective amount of a compound according to claim 1 to patient.

25. A method of inhibiting DNA-PK in vitro or in vivo, comprising contacting a cell with an effective amount of a compound according to of claims 1.
